(19) United States
(12) Patent Application Publication
(10) Pub. No.: US 2026/0297414 A1
(43) Pub. Date: Oct. 1, 2026

(54) EMULSIFIER AND PREPARATION METHOD THEREFOR, AND OIL-BASED DRILLING FLUID

(71) Applicants: China Petroleum & Chemical Corporation, Beijing (CN); Sinopec Oilfield Service Corporation, Beijing (CN); Sinopec Shengli Petroleum Engineering Co., Ltd, Dongying, Shandong (CN); Drilling Fluid Technology Service Center of Sinopec Shengli Petroleum Engineering Co., Ltd, Dongying, Shandong (CN)

(72) Inventors: Ke LI, Dongying, Shandong (CN); Chengjun WANG, Dongying, Shandong (CN); Xuxin WAN, Dongying, Shandong (CN); Haibin LI, Dongying, Shandong (CN); Gongrang LI, Dongying, Shandong (CN); Gaoqun HU, Dongying, Shandong (CN); Yunfeng LUO, Dongying, Shandong (CN); Xiaoxuan ZHOU, Dongying, Shandong (CN); Bo LI, Dongying, Shandong (CN)

(73) Assignees: China Petroleum & Chemical Corporation, Beijing (CN); Sinopec Oilfield Service Corporation, Beijing (CN); Sinopec Shengli Petroleum Engineering Co., Ltd, Dongying, Shandong (CN); Drilling Fluid Technology Service Center of Sinopec Shengli Petroleum Engineering Co., Ltd, Dongying, Shandong (CN)

(21) Appl. No.: 19/472,122

(22) PCT Filed: Apr. 26, 2024
(86) PCT No.: PCT/CN2024/090081

§ 371 Date: Oct. 3, 2025

(30) Foreign Application Priority Data
Apr. 27, 2023 (CN) 202310477151.5

(51) Int. Cl. C09K 8/36; C07C 303/28; C07C 309/21; E21B 21/06

(52) U.S. Cl. CPC C09K 8/36; C07C 303/28; C07C 309/21; E21B 21/062

(57) ABSTRACT

Described are an emulsifier and a preparation method therefor, and an oil-based drilling fluid. The emulsifier has a polyene polyamine skeleton and a hydrophobic group connected to the skeleton, and part of the polyene polyamine skeleton is connected to a sulfonic acid group. The molar ratio of a sulfur element to a nitrogen element in the emulsifier is 0.02-0.4:1. In the emulsifier, the part of the emulsifier that does not contain the sulfonic acid group can function as a main emulsifier, and the part of the emulsifier that contains the sulfonic acid group has suitable hydrophilicity and can function as an auxiliary emulsifier. A composite emulsifier formed based on compounds of the same structure and type can achieve closer fusion to form a compact composite interfacial film, and has an excellent emulsifying effect and good electrical stability and thermal stability.

### CROSS-REFERENCE TO RELATED APPLICATIONS

The application claims the benefit of the China patent application No. “202310477151.5”, filed on Apr. 27, 2023, the content of which is specifically and entirely incorporated herein by reference.

### TECHNICAL FIELD

The present invention relates to the technical field of oil-based drilling fluid emulsifiers, in particular to an emulsifier and a preparation method therefor, and an oil-based drilling fluid.

### BACKGROUND ART

The oil-based drilling fluids have excellent properties such as strong suppression, anti-pollution, lubrication, salt resistance and low reservoir damage, and are used more widely in water-sensitive formations, shale formations, deep wells, giant thick salt paste layers, salt-stone strata and the like. The emulsifier is one of the most critical treatment agents for the oil-based drilling fluids, it mainly serves to ensure the emulsion stability of the water-in-oil drilling fluids, thereby improving the rheological property of the oil-based drilling fluids. Currently, the emulsifier composition of oil-based drilling fluids is mostly composed of a main emulsifier and an auxiliary emulsifier, which cooperate with the hydrophilic surfactants based on the different structures, however, when the surfactants with different structures form an interfacial film, the integration degree is poor due to the diverse structures, thus it is not beneficial to the strength maintenance and stabilization of the emulsion. In addition, the existence of a main emulsifier and an auxiliary emulsifier will also increase the difficulty of the on-site operations.

CN115785920A discloses an emulsifier for water-in-oil emulsion drilling fluid, which is prepared by carrying out cyclization reaction by selecting fatty acid and organic amine as the reaction monomer, it is ensured that the emulsifier can be kept stable under the high-temperature environment by taking advantage of the five-membered heterocyclic ring structure in the molecular structure of the emulsifier, but the demulsification voltage of the emulsifier is below 400V. CN105907382A discloses an emulsifier based on an oil-based drilling fluid, the emulsifying agent contains tall oil fatty acid, polyamine, chloroactic acid amide, surfactants and organic solvents, it is compounded by adding the surfactants, but the invention merely discloses the temperature resistance of the emulsifier after an aging process at 150° C.

Therefore, given that the bottom hole temperatures are at record highs and the requirements on high-temperature resistance of the oil-based/synthetic-based drilling fluid emulsifier are gradually prominent, it is urgent to solve the technical problems concerning how to further improve the electrical stability and temperature resistance of the oil-water emulsion system in the oil-based drilling fluid and reduce the operation difficulty.

### SUMMARY OF THE INVENTION

The present invention aims to overcome the defects in the prior art with respect to the poor electrical stability and temperature resistance of the oil-water emulsion systems, and provides an emulsifier and a preparation method therefor, and an oil-based drilling fluid, the emulsifier has excellent emulsifying properties, desirable electrical stability and strong temperature resistance.

In order to achieve the above objects, the first aspect of the present invention provides an emulsifier comprising a polyene polyamine skeleton and a hydrophobic group connected to the skeleton, and part of the polyene polyamine skeleton is connected to a sulfonic acid group, wherein the molar ratio of a sulfur element to a nitrogen element in the emulsifier is 0.02-0.4:1.

The second aspect of the present invention provides a preparation method for the emulsifier, the preparation method comprises the following steps: contacting a main emulsifier with a sulfonating agent to carrying out the sulfonation reaction to introduce a sulfonic acid group into a portion of the main emulsifier; wherein the main emulsifier has a polyene polyamine skeleton and a hydrophobic group connected to the skeleton; the molar ratio of the sulfonating agent to the main emulsifier calculated in terms of polyene polyamine skeleton is (0.1-0.5):1.

The third aspect of the present invention provides an emulsifier produced with the aforementioned preparation method.

The fourth aspect of the present invention provides an oil-based drilling fluid, the oil-based drilling fluid comprises a base oil, and a treatment agent comprising the emulsifier according to the first aspect or the third aspect.

The present invention provides an emulsifier comprising a compound having a polyene polyamine skeleton and a hydrophobic group connected to the skeleton, and part of the polyene polyamine skeleton is connected to a sulfonic acid group; a portion of the emulsifier that does not contain a sulfonic acid group can function as a main emulsifier, the hydrophobic group connected to the skeleton enables the main emulsifier to have a strong lipophilicity, and reduces the oil-water interfacial tension by forming an absorbent film having a certain strength at the oil-water interface; a portion of the emulsifier contains a sulfonic acid group exhibits an appropriate hydrophilicity, and can function as an auxiliary emulsifier, the composite emulsifier consisting of the compounds based on the same structure and type can be more closely blended to form a dense composite interfacial film thereby enhancing the emulsifying effect. The emulsifier has an appropriate molar ratio of a sulfur element to a nitrogen element, it indicates that the emulsifier has an appropriate content of a sulfonic acid group; when a molar ratio of sulfur element to nitrogen element is controlled to be within the aforementioned range, it can produce the synergistic effect of a main emulsifier and an auxiliary emulsifier, effectively reduce oil-water interfacial tension, form a stable interfacial film, prevent the coalescence of water droplets, such that the oil-water emulsion system becomes stable. The emulsifier as an oil-based drilling fluid emulsifier may cause that the demulsification voltage of an oil-water emulsion system reaches 1,089V, the maximum demulsification voltage after an aging process at 180° C. and 200° C. can reach 974V and 648V respectively, indicating that the emulsifier provided by the present invention has significantly superior emulsifying stability compared to the existing composite emulsifier system.

The preparation method of the emulsifier provided in the present invention, can introduce sulfonic acid group into polyamine compound through the sulfonation reaction to obtain the sulfonate substitution of a part of the corresponding structure, it synthesizes the main emulsifier and the auxiliary emulsifier having the same structure type through the “one-pot method”, thereby reducing the production costs and the steps and improving the reaction efficiency.

### BRIEF DESCRIPTION OF THE DRAWINGS

FIG. 1 illustrates an infrared absorption spectrogram of the main emulsifier prepared in Example 1 of the present invention;

FIG. 2 shows a mass spectrogram of the main emulsifier prepared in Example 1 of the present invention;

FIG. 3 illustrates an infrared absorption spectrogram of the emulsifier A1 prepared in Example 1 of the present invention;

FIG. 4 shows a mass spectrogram of the emulsifier A1 prepared in Example 1 of the present invention.

### DESCRIPTION OF THE PREFERRED EMBODIMENT

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point values of the various ranges, and the individual point values may be combined with one another to produce one or more new numerical ranges, which should be deemed to have been specifically disclosed herein.

The first aspect of the present invention provides an emulsifier comprising a polyene polyamine skeleton and a hydrophobic group connected to the skeleton, and part of the polyene polyamine skeleton is connected to a sulfonic acid group, wherein the molar ratio of a sulfur element to a nitrogen element in the emulsifier is 0.02-0.4:1.

According to the invention, part of the polyene polyamine skeleton contains a sulfonic acid group, that is, part of the polyene polyamine skeleton is connected to a sulfonic acid group, and the remaining part of the polyene polyamine skeleton is not connected with a sulfonic acid group, under the circumstance, the compound that does not contain a sulfonic acid group in the skeleton can function as a main emulsifier, the compound containing a sulfonic acid group in the remaining part of the skeleton can function as an auxiliary emulsifier, the main emulsifier and the auxiliary emulsifier formed based on the same structure and type of compounds can be blended more closely. In the prior art, when the surfactants with different structures and hydrophilicities are combined as an emulsifier, because the different surfactants have diverse structures, it is difficult to integrate the surfactants, the stability is poor, the emulsifier generally need to be prepared through the on-site operation, thereby increasing the difficulty of the on-site operation.

In the invention, the molar ratio of a sulfur element to a nitrogen element in the emulsifier is 0.02-0.4:1, for example, it may be 0.02:1, 0.03:1, 0.04:1, 0.05:1, 0.06:1, 0.07:1, 0.08:1, 0.09:1, 0.1:1, 0.11:1, 0.12:1, 0.13:1, 0.14:1, 0.15:1, 0.16:1, 0.17:1, 0.18:1, 0.19:1, 0.2:1, 0.22:1, 0.24:1, 0.26:1, 0.28:1, 0.3:1, 0.35:1, 0.4:1, and the other typical and non-limiting molar ratio or a random value within the range consisting of any two molar ratios. By controlling the suitable molar ratio of the sulfur element to the nitrogen element in the emulsifier, which indicates that the emulsifier has an appropriate relative content of polyene polyamine skeleton and sulfonic acid group, the interaction of the polyene polyamine skeleton and the sulfonic acid group can effectively reduce the oil-water interfacial tension, form a stable interfacial film, prevent the coalescence of water drops and enable the oil-water emulsification system to become stable. Preferably, the molar ratio of a sulfur element to a nitrogen element in the emulsifier is 0.08-0.3:1, further preferably 0.1-0.2:1. In the case of preferably, the emulsifying effect of the emulsifier can be further enhanced, the electrical stability and the thermal stability of the emulsifier can be improved.

In the invention, an organic element analyzer is used for testing the mass fractions of a sulfur element and a nitrogen element in the emulsifier, the molar ratio of the sulfur element to the nitrogen element is then obtained by calculation.

According to the invention, preferably, the demulsification voltage of the emulsifier in a standard oil-based emulsion under the condition of a dosage of 5 wt % is within the range of 50-400V, more preferably within the range of 80-280V, further preferably within the range of 200-280V, wherein the standard oil-based emulsion is composed of 5 # white oil and CaCl₂ aqueous solution having a concentration of 25 wt %, the volume ratio of oil-water is 80:20; the dosage refers to the mass fraction of the addition amount of an emulsifier based on the total mass of oil and water. The inventors of the present invention have discovered in researches that the emulsifier has a suitable demulsification voltage in a standard oil-based emulsion, the demulsification voltage within the preferred range is favorable for application of an emulsifier in the oil-based drilling fluid in practice, and the emulsifier has excellent emulsification performance. The demulsification voltage of an emulsifier in standard oil-based emulsions is not the higher the better, because the drilling fluids in practice have a relatively complicated composition, when the demulsification voltage of an emulsifier in the standard oil-based emulsions is too high, the emulsifier exhibits an excessively high polarity in the actual oil-based drilling fluids, the formed oil droplets are too tiny, which may result in increased plastic viscosity and dynamic shear force, and poor electrical stability and thermal stability.

According to some preferred embodiments of the present invention, the emulsification ratio of the emulsifier in the standard oil-based emulsion is larger than or equal to 90%, more preferably within the range of 95-100%.

According to the invention, the molar ratio of the hydrophobic group to the polyene polyamine skeleton is preferably (1-4):1, for example, it may be 1:1, 2:1, 3:1, 4:1, and the other typical and non-limiting molar ratio or a random value within the range consisting of any two molar ratios. More preferably, the molar ratio of the hydrophobic group to the polyene polyamine skeleton is (2-3):1.

According to the present invention, preferably, the polyene polyamine skeleton is present in an amount of 5-20 wt %, more preferably 10-15 wt %, based on the total amount of the emulsifier.

According to the invention, the molar ratio of the sulfonic acid group to the polyene polyamine skeleton is preferably (0.1-1.2):1, more preferably (0.3-0.6):1. In the above preferred circumstances, it is advantageous to further improve the emulsifying effect and stability of the emulsifier.

According to some preferred embodiments of the invention, the emulsifier contains C, H, N, S and O elements.

Further preferably, the content of C element is 50-75 wt %, preferably 55-72 wt %; the content of H element is 5-15 wt %, preferably 8-13 wt %; the content of N element is 2-8 wt %, preferably 2.5-5.5 wt %; the content of S element is 0.5-2 wt %, preferably 0.8-1.5 wt %, and the content of O element is 10-25 wt %, preferably 11-20 wt %, based on the total amount of the emulsifier. In the invention, an organic element analyzer is used for testing the elemental composition in the emulsifier. It is understandable that the emulsifier may further comprise other elements, the present invention does not impose the specific limitations thereto.

In the invention, the polyene polyamine skeleton is at least one selected from the group consisting of diethylene triamine skeleton, triethylene tetramine skeleton and tetraethylene pentamine skeleton. It is understandable that at least one of the H atoms connected with the N atoms in the polyene polyamine skeleton is replaced by a hydrophobic group, so that the hydrophobic group is connected to the polyethylene polyamine skeleton. Preferably, the polyene polyamine skeleton is selected from diethylene triamine and/or triethylene tetramine.

In the invention, the hydrophobic group is connected with the polyene polyamine skeleton via a covalent bond, preferably, the hydrophobic group is connected with the N atom at the end of the polyene polyamine skeleton, under the preferred circumstance, the hydrophobic group is concentrated outside the compound structure and forms a synergistic action with the hydrophilic polyene polyamine skeleton inside, it is conducive to further improving the emulsifying effect of the emulsifier and enhancing the electric stability and thermal stability of the emulsifying system.

In the invention, a hydrophobic group refers to that the entire structural unit exhibits hydrophobicity (lipophilicity), and the specific composition of the hydrophobic group is not particularly limited in the invention. Preferably, the hydrophobic group contains at least one hydrophobic functional group. The present invention has a wide selection range for the hydrophobic functional group, preferably, the hydrophobic functional group is at least one selected from the group consisting of alkyl, alkenyl, alkynyl, aryl and ester group. For example, the hydrophobic group may be straight-chain or branched C3-C18 alkyl, preferably any one of propyl, butyl, pentyl, hexyl, heptyl, nonyl, decyl, dodecyl, tetradecyl, hexadecyl, octadecyl, and isomers thereof, and any one of carbon atoms or hydrogen atoms of the alkyl may be further replaced by a substituent, for example, the substituent may be any one of ester group, hydroxyl and phenyl. For instance, the terminal carbon atom of the n-decyl may be substituted with a methyl ester group. The hydrophobic group may further contain a hydrophilic group such as hydroxyl, it is not limited in the invention, so long as the structural unit exhibits hydrophobicity as a whole.

According to some preferred embodiments of the invention, the hydrophobic group is

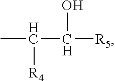

wherein R₄ is selected from H or C5-C16 alkyl; R₅ is selected from substituted or unsubstituted C5-C16 alkyl, alkoxy, aryl or phenoxy. The C5-C16 alkyl group is preferably any one of propyl, butyl, pentyl, hexyl, heptyl, nonyl, decyl, dodecyl, tetradecyl, hexadecyl, octadecyl, and isomers thereof, any one of carbon atoms or hydrogen atoms of the alkyl group may be further replaced by a substituent, for example, the substituent may be any one of ester group, hydroxyl and phenyl.

The invention does not impose special requirements on the particular structure of the substituent containing sulfonic acid group, as long as a part of polyene polyamine skeleton comprises the sulfonic acid group, the polyene polyamine skeleton can produce the synergistic emulsification effect with the compound that does not contain a sulfonic acid group; in order to further improve the emulsification performance of the emulsifier, according to some preferred embodiments of the invention, the structure of the sulfonic acid group is represented by

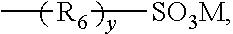

wherein R₆ is selected from substituted or unsubstituted C1-C8 alkylidene; y is 0 or 1; and M is selected from Na, K or ammonium group. For example, the alkylidene may be any one of methylene, ethylidene, propylidene, butylidene, and isomers thereof, and H atom on at least one C atom in the alkylidene may be replaced by a substituent, for instance, the substituent may be any one of hydroxyl, ester group and ether group.

Preferably, M is selected from alkali metal or ammonium group, preferably Na, K or ammonium group.

In the invention, y in formula (III) is 0 or 1, it is understandable when y is 0, it means that the substituent comprising a sulfonic acid group does not contain an alkylidene, under the circumstance, the sulfonic acid group is directly connected with the N atom.

According to some preferred embodiments of the invention, the emulsifier comprises a compound represented by formula (I);

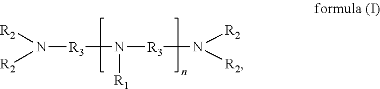

wherein each R₁ is independently selected from H or the sulfonic acid group, each R₂ is independently selected from H or the hydrophobic group, and at least one R₂ is not H; each R₃ is independently selected from substituted or unsubstituted C1-C3 alkylidene; n is a positive integer between 1 and 3.

According to the invention, in formula (I), R₁ in the number n of repeated structural units can be the same or different, and are respectively and independently selected from H or the sulfonic acid group, such that a part of polyene polyamine skeleton is connected with a sulfonic acid group, and the molar ratio of sulfur element to nitrogen element meets the aforementioned requirement.

For example, when the polyene polyamine skeleton is triethylene tetramine, formula (I) may be represented by

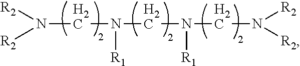

each R₁ is independently selected from H or the sulfonic acid group; when the polyene polyamine sulfone is diethylene triamine, the formula (I) can be represented by

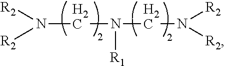

each R₁ is independently selected from H or the sulfonic acid group, and R₁ has the same definition range in the preceding paragraphs.

The second aspect of the present invention provides a preparation method for the emulsifier, the preparation method comprises the following steps: contacting a main emulsifier with a sulfonating agent to carrying out the sulfonation reaction to introduce a sulfonic acid group into a portion of the main emulsifier; wherein the main emulsifier has a polyene polyamine skeleton and a hydrophobic group connected to the skeleton; the molar ratio of the sulfonating agent to the main emulsifier calculated in terms of polyene polyamine skeleton is (0.1-0.5):1.

The polyene polyamine skeleton and the hydrophobic group have the same definitions as in the first aspect, the definitions are not repeatedly described herein.

According to some preferred embodiments of the invention, the content of polyene polyamine skeleton is 40-100 wt %, more preferably 50-90 wt %, based on the total amount of the main emulsifier.

According to some preferred embodiments of the present invention,
the main emulsifier comprises a compound with the structure represented by formula (i);

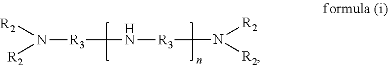

Wherein each R₂ is independently selected from H or the hydrophobic group, and at least one R₂ is not H, each R₃ is independently selected from substituted or unsubstituted C1-C3 alkylidene; n is a positive integer between 1 and 3. R₂ and R₃ therein have the same definitions as those in the first aspect.

The source of the main emulsifier is not particularly required in the invention, it can be prepared with any chemical reaction method known in the field.

According to some preferred embodiments of the invention, the preparation method for the main emulsifier comprises the following steps: subjecting the polyene polyamine and an epoxy compound to the ring-opening reaction;
wherein the epoxy compound has a structure represented by formula (a),

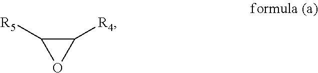

wherein R₄ and R₅ have the same definitions as those in the first aspect.

According to the present invention, in step (1), the hydrophobic group is introduced into the polyene polyamine by means of the ring-opening reaction of the polyene polyamine with an epoxy compound.

According to the invention, the structure of the polyene polyamine may be represented as

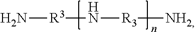

wherein n and R₃ have the same definition as those in the first aspect.

According to some preferred embodiments of the invention, the polyene polyamine is selected from diethylene triamine and/or triethylene tetramine.

According to some preferred embodiments of the invention, preferably, the epoxy compound is at least one of methyl 9,10-epoxyoctadecanoate (cas: 6084-76-0), 1,2-epoxyoctadecane (cas: 7390-81-0) and C10-16-alkyl glycidyl ether (cas: 68081-84-5). The polyethylene polyamine and epoxy compound are commercially available, or may be prepared with a known method, the present invention does not impose the particular limitations thereto.

According to the invention, the molar ratio of the polyene polyamine to the epoxy compound is preferably 1:(1-5), for example, it may be 1:1, 1:1.5, 1:2, 1:2.5, 1:3, 1:3.5, 1:4, 1:4.5, 1:5, and the other typical and non-limiting molar ratio or a random value within the range consisting of any two molar ratios. Preferably, the molar ratio of the polyene polyamine to the epoxy compound is 1:(1.5-3.5).

In the present invention, a polyene polyamine and an epoxy compound can be put into a reactor to carry out the ring-opening reaction. The conditions of the ring-opening reaction are not particularly limited in the invention, as long as the conditions may facilitate the ring-opening reaction of the epoxy compound, those skilled in the art can select the conditions according to actual needs. Preferably, the conditions of the ring-opening reaction comprise: the reaction temperature within the range of 60-140° C., more preferably within the range of 80-120° C., and the reaction time within the range of 5-15 h, more preferably within the range of 6-12 h.

Preferably, the ring-opening reaction is carried out under the stirring conditions, and the stirring conditions are not particularly limited in the invention, as long as the reaction can be performed smoothly.

In the invention, the ring-opening reaction may be carried out in the presence of a catalyst, and the catalyst is not particularly limited in the invention, any catalyst known in the field to promote the ring-opening reaction of an epoxy compound may be applied in the invention. For example, the catalyst may be an organic acid and/or an inorganic acid, preferably at least one of sulfuric acid, phosphoric acid, acetic acid, oxalic acid, trifluoroacetic acid, p-toluenesulfonic acid and sulfamic acid, further preferably at least one of sulfuric acid, phosphoric acid, acetic acid and oxalic acid.

Preferably, the molar ratio of the catalyst to the epoxy compound is (0.001-0.02):1, more preferably (0.003-0.015):1.

In the invention, the product of the ring-opening reaction may be directly used as the main emulsifier to continuously perform the sulfonation reaction, or the product mixture obtained from the ring-opening reaction may be initially purified to remove unreacted raw materials which may be present by using various means, the sulfonation reaction may be subsequently performed. The present invention has no special requirement, as long as the product contains the compound with the desired structure, those skilled in the art can make a choice according to the actual needs.

The invention has no special requirements on the specific type of the sulfonating agent, as long as the sulfonic acid group is introduced into a part of the main emulsifier, it can be selected by those skilled in the art according to the actual situation. The source of the sulfonating agent is not particularly limited in the invention, the sulfonating agent may be commercially available or prepared with the chemical reaction method known in the field.

According to some preferred embodiments of the invention, the sulfonating agent is a sulfonate and/or an alkyl sulfonate lactone.

Preferably, the sulfonate has a structure represented by formula (ii),

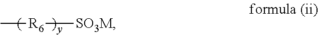

wherein X is a halogen atom, preferably Cl or Br; R₆ is selected from substituted or unsubstituted C1-C8 alkylidene; preferably C2-C4 alkylidene, for example, it may be any one of methylene, ethylidene, propylidene, butylidene, pentylidene, hexylidene, heptylidene and octylidene or isomers thereof; y is 0 or 1; M is selected from Na, K or ammonium group.

According to some preferred embodiments of the invention, the sulfonating agent is at least one of sodium 3-chloro-2-hydroxypropane sulfonate (cas: 126-83-0), 1,3-propane sultone (cas: 1120-71-4) and/or 1,4-butane sultone (cas: 1633-83-6), more preferably 1,3-propane sultone and/or 1,4-butane sultone.

According to the invention, in order to expedite the sulfonation reaction and prevent the formation of an inorganic acid, the sulfonation reaction is preferably carried out in the presence of an acid-binding agent. The specific type of the acid-binding agent is not particularly limited in the invention, it may be an acid-binding agent conventional in the field, for example, an organic alkali or an inorganic alkali, preferably an inorganic alkali, it may be, for instance, at least one of sodium carbonate, potassium carbonate, sodium hydroxide and potassium hydroxide.

According to the invention, the molar ratio of the acid-binding agent to the sulfonating agent is preferably (0.5-2.5):1, more preferably (0.5-2):1. Under the preferable condition, the acid-binding agent is favorable for effectively binding the acidic byproduct generated in the reaction process and promoting the proceeding of the forward reaction, in the meanwhile, the acid-binding agent is alkaline salt, its addition in a suitable amount can effectively reduce the formation of salt content on the basis of not influencing the reaction, and avoid the defects that the reaction product has an overhigh viscosity and is difficult to pour and use on site.

According to the present invention, the molar ratio of the sulfonating agent to the main emulsifier calculated in terms of polyene polyamine skeleton is preferably (0.1-0.5):1, more preferably (0.2-0.4):1. By adopting the preferred embodiment, the polyene polyamine skeleton in the prepared emulsifier can be controlled to have a suitable sulfonation degree, such that the emulsifying effect of the emulsifier can be further enhanced, and the electric stability and thermal stability of an emulsifying system can be improved.

The specific conditions of the sulfonation reaction are not particularly limited in the invention, as long as the conditions are favorable for the sulfonation reaction, those skilled in the art can select the conditions according to the actual needs. Preferably, the sulfonation reaction conditions comprise: the reaction temperature within the range of 80-120° C., and the reaction time within the range of 3-5 h.

In the invention, the sulfonation reaction can also be carried out in the presence of a solvent, and the solvent may be selected from a wide range in the invention, and the solvent may be an organic solvent commonly used in the field, for example, at least one of methanol, ethanol, n-octanol, iso-octanol, dimethyl formamide, dimethyl sulfoxide and ethylene glycol. The dosage of the solvent is not particularly limited in the invention, as long as the reactants can be sufficiently dispersed, the solvent can be selected by those skilled in the art according to actual needs.

According to some preferred embodiments of the invention, the preparation method further comprises: removing the solvent from the mixture obtained by the sulfonation reaction. In the present invention, the solvent in the product mixture may be removed by using various methods, preferably the reduced pressure distillation method. For example, a method for removing the solvent from the mixture obtained by the sulfonation reaction comprises: cooling the mixture obtained by the sulfonation reaction, and then carrying out the reduced pressure distillation. Preferably, the reduced pressure distillation is performed under a pressure of 1-5 kPa, more preferably 1.2-2 kPa, and a temperature within the range of 40-80° C., preferably within the range of 45-60° C. Unless otherwise specified in the invention, the pressure refers to an absolute pressure.

According to a particularly preferred embodiment of the invention, the preparation method for the emulsifier comprises the following steps:
(1) Carrying out the ring-opening reaction on the polyene polyamine and an epoxy compound;
Wherein the epoxy compound has a structure represented by formula (a),

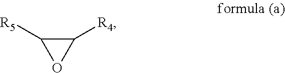

wherein R₄ and R₅ have the same definitions as those in the first aspect;
(2) Contacting the product of the ring-opening reaction with a sulfonating agent to carry out the sulfonation reaction;
Wherein the molar ratio of the polyene polyamine, the epoxy compound and the sulfonating agent is 1:(1-5):(0.2-0.5).

It should be noted that the product obtained from the aforementioned preparation method is generally present in the form of a mixture: step (1) carrying out the ring-opening reaction on the polyene polyamine and an epoxy compound to obtain a main emulsifier having a polyene polyamine skeleton and a hydrophobic group connected to the skeleton, a sulfonic acid group is then introduced into the part of polyene polyamine skeleton of the main emulsifier through the sulfonation reaction to obtain a sulfonated product of the main emulsifier, the composite emulsifier consisting of the main emulsifier and the sulfonated product of the main emulsifier based on the same polyene polyamine skeleton structure can be blended more closely, the interaction of the main emulsifier and the sulfonated product can effectively reduce the oil-water interfacial tension, form a stable interfacial film, prevent the coalescence of water drops, and enable an oil-water emulsification system to become stable. The emulsifier serving as an oil-based drilling fluid emulsifier has excellent emulsifying effect, desirable electrical stability and thermal stability, can be effectively applied in the oil-based drilling fluids having various oil-water ratios and different types of base oil, it is suitable for emulsification of oil-based drilling fluids such as gas oil, white oil and diesel.

The third aspect of the present invention provides an emulsifier produced with the aforementioned preparation method.

The fourth aspect of the present invention provides an oil-based drilling fluid, the oil-based drilling fluid comprises a base oil, and a treatment agent comprising the aforementioned emulsifier.

In the invention, the type of base oil in the oil-based drilling fluid can be selected from a wide range, the base oil can be provided by the oil phase conventionally used in the field, it can be performed by those skilled in the art according to the actual needs. Preferably, the base oil is at least one of gas oil, white oil and diesel, more preferably white oil.

According to some preferred embodiments of the invention, the content of said emulsifier is 2-10 parts by weight, more preferably 3-5 parts by weight, based on 100 parts by weight of the base oil.

According to the invention, for example, the treating agent may further comprise at least one of an organic soil, a plugging agent, a weighting agent, a wetting agent, an alkaline regulator and a filtrate reducer. Those skilled in the art can select the materials according to actual needs, and the invention does not impose specific limitations thereto.

According to some preferred embodiments of the invention, the organic soil may be at least one of organically modified bentonite, organically modified sepiolite, organically modified palygorskite, for example. The source of the organic soil in the invention is not particularly limited, it may be commercially available.

The source of the filtrate reducer is not particularly limited in the invention, the filtrate reducer may be the commercially available product well-known among those skilled in the art. Preferably, the filtrate reducer is at least one of natural asphalt, oxidized asphalt and styrene butadiene rubber.

According to some preferred embodiments of the invention, the weighting agent may be at least one of barite, micro-manganese ore powder and iron ore powder.

According to some preferred embodiments of the invention, the alkaline regulator may be calcium oxide and/or sodium carbonate, preferably calcium oxide.

In the invention, the dosages of the filtrate reducer, the alkaline regulator and the organic soil can be selected according to actual needs, and the dosages are not particularly limited in the invention. According to some preferred embodiments of the invention, the dosage of the filtrate reducer is 2-6 parts by weight, the dosage of the alkaline regulator is 0.5-2 parts by weight, and the dosage of the organic soil is 0.5-3 parts by weight, based on 100 parts by weight of the base oil.

The present invention will be described in detail below with reference to examples.

Unless otherwise specified in the following examples and comparative examples, the raw materials in use were all commercially available.

In the invention, the method for testing the demulsification voltage and the emulsification rate of an emulsifier in the standard oil-based emulsion comprised the following steps: the emulsifier and 5 # white oil were stirred at high speed for 30 minutes under the condition of 10,000 r/min to dissolve the emulsifier in the oil, the CaCl₂ aqueous solution with a concentration of 25 wt % was then added according to the oil-water volume ratio of 80:20, and stirred at a high speed of 10,000 r/min for 2 h, the emulsion to be detected was obtained. The addition amount of the emulsifier was 5 wt %, based on the total mass of the oil and the water.

The electrical stability test: a probe of an electrical stability tester was placed in the emulsion to be tested, the measurement results were recorded, the average demulsification voltage value of the two measurement results were calculated, wherein the difference of the two readings was not more than 5%.

The emulsification rate test: the emulsion to be detected was subjected to an aging process at 150° C. for 16 h, then cooled to room temperature, stirred at a high speed of 10,000 r/min for 20 min, the emulsion was poured into a 500 mL measuring cylinder, subjected to standing still for 24 h, and the volume of the separated oil phase was read. The emulsification rate (W) was calculated according to the following equation:

$$W = ( {V}_{0} - V ) / {V}_{0} \times 100 \% ;$$

In the above formula, V₀ denoted the volume of an emulsion, and the unit was mL; W denoted the emulsification rate, the unit was %; V denoted the volume of separated oil layer, and the unit was mL.

In the invention, an organic element analyzer with an instrument model Elementar Unicube was used for measuring the contents of C, H, O, N, S elements in the product. The molar ratio of the S element and the N element in the emulsifier was obtained by further calculation.

In the invention, the infrared absorption spectrum was tested by a Fourier infrared spectrometer, and the test mode was an Attenuated Total Reflectance (ATR) mode.

In the invention, the mass spectrometry was tested in a matrix-assisted laser desorption ionization time-of-flight mass spectrometry (MALDI-TOF/TOF), and the test conditions comprised: dissolved with methanol, the matrix was CHCA (Cyano-4-hydroxycinnamic acid).

### Example 1

(1) 20 mol of diethylene triamine and 38 mol of methyl 9,10-epoxyoctadecanoate (cas: 6084-76-0, commercially available, with the purity of 75%) were added into a double-neck round-bottom flask with a reflux condenser pipe, the materials were mixed and stirred uniformly, 0.2 mol of para-toluenesulfonic acid was added and blended, and then stirred and heated to 90° C. for performing the reaction for 7.5 hours, a main emulsifier was obtained.

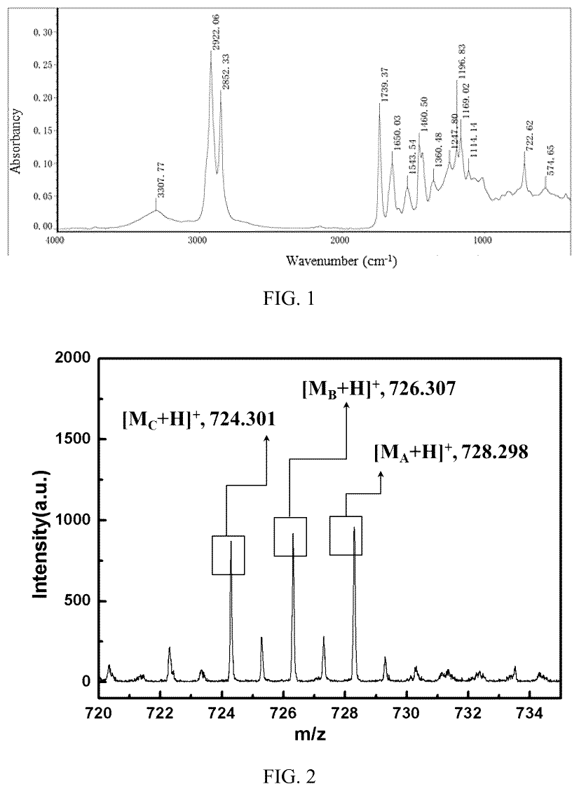

Apart of main emulsifier was taken and separated by the silica gel column chromatography (the ratio of petroleum ether to ethyl acetate was 2:1-1:5), a solvent was distilled out by a rotary evaporator to obtain a purified product, which was then subjected to an infrared absorption spectrum analysis, the results were shown in FIG. 1, the infrared absorption spectrum showed that the absorption peak belonging to epoxy bonds at 1080-1160 cm⁻¹ was disappeared, the characteristic peak belonging to hydroxyl groups at 3200-3500 cm⁻¹ was very obvious, it demonstrated that the ring-opening reaction of diethylene triamine and epoxy fatty acid methyl ester was carried out. The mass spectrometry was performed on the main emulsifier, the results were shown in FIG. 2, the structure corresponding to characteristic peaks with the mass-to-charge ratios of 724.301, 726.307 and 728.298 was diethylenetriamine substituted by dihydroxy fatty acid methyl ester, the multimodal signals were derived from the presence of linoleic acid methyl ester and oleic acid methyl ester in the methyl 9,10-epoxyoctadecanoate component, the epoxy methyl oleate and epoxy methyl linoleate can be generated after epoxidation of linoleic acid methyl ester and oleic acid methyl ester. The above results proved that the main emulsifier was successfully synthesized, it was demonstrated with reference to the results of mass spectrometry and infrared adsorption spectrum that the main emulsifier had a structure represented by formula (i), wherein two groups R₂ were

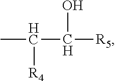

R₄ was n-octyl carbomethoxy, and R₅ was n-octyl. The molar ratio of the hydrophobic group to the diethylene triamine skeleton was 2:1.
(2) The product obtained in step (1), 6 mol of sodium 3-chloro-2-hydroxy propane sulfonate and 150 mL of ethanol were uniformly mixed, 6 mol of potassium carbonate was added and mixed, the mixture was heated to 80° C., stirred and subjected to reaction for 3 hours, a second intermediate was obtained;
(3) The second intermediate obtained in step (2) was cooled to room temperature, then reduced pressure distilled under the pressure of 1.5 kPa and the temperature of 50° C. to remove solvent ethanol, an emulsifier A1 was prepared.

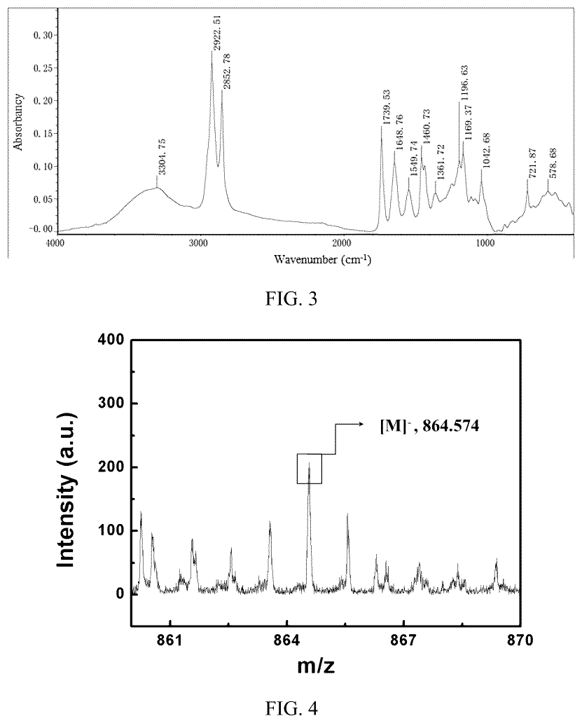

The emulsifier A1 was separated by the silica gel column chromatography (the ratio of dichloromethane to methanol was 1:1-1:8), the solvent was distilled out by a rotary evaporator to obtain a purified product, which was then subjected to an infrared absorption spectrum test, the infrared absorption spectrum was shown in FIG. 3, the infrared absorption spectrum showed that the characteristic absorption peak belonging to hydroxyl at 3200-3500 cm⁻¹ became large, which indicated that the content of hydroxyl was increased, and the characteristic peak belonging to sulfonate group at 1042.68 cm⁻¹ appeared, which demonstrated that sulfonate group existed in the structure. The mass spectrometry results were shown in FIG. 4, the structure corresponding to the characteristic peak with the mass-to-charge ratio of 864.574 was diethylene triamine substituted by the dihydroxy fatty acid methyl ester into which a sulfonic acid group was introduced, it was indicated with reference to the results of FIG. 3 and FIG. 4 that a sulfonic acid group was introduced into diethylene triamine skeleton of the main emulsifier. The elemental composition was analyzed by an organic element analyzer, wherein the content of N element was 4.92 wt %, the content of C element was 65.4 wt %, the content of H element was 10 wt %, the content of S element was 1.15 wt %, and the content of O element was 16.95 wt %. The molar ratio of S element to N element in the emulsifier was 0.23:1. The content of the diethylene triamine skeleton was 12 wt % based on the total amount of the emulsifier. The molar ratio of the sulfonic acid group to the polyene polyamine skeleton was 0.31:1.

The emulsification rate and demulsification voltage of the emulsifier A1 in standard oil-based emulsions were shown in Table 1.

### Example 2

(1) 20 mol of diethylene triamine and 38 mol of methyl 9,10-epoxyoctadecanoate (cas: 6084-76-0) were added into a double-neck round-bottom flask with a reflux condenser pipe, the materials were mixed and stirred uniformly, 0.25 mol of sulfuric acid was added and blended, and then stirred and heated to 100° C. for performing the reaction for 8 hours, a main emulsifier was obtained. The molar ratio of the hydrophobic group to the diethylene triamine skeleton was 2:1.
(2) The main emulsifier, 6 mol of sodium 3-chloro-2-hydroxy propane sulfonate and 200 mL of isooctanol were uniformly mixed, 6 mol of sodium hydroxide was added and mixed, the mixture was heated to 90° C., stirred and subjected to reaction for 4 hours;
(3) The product obtained in step (2) was cooled to room temperature, then reduced pressure distilled under the pressure of 1.2 kPa and the temperature of 50° C. to remove solvent isooctanol, an emulsifier A2 was prepared.

The emulsifier A2 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.1 wt %, the content of C element was 64.2 wt %, the content of H element was 11.2 wt %, the content of S element was 1.2 wt %, and the content of O element was 16.7 wt %. The molar ratio of S element to N element in the emulsifier was 0.1:1. The content of the diethylene triamine skeleton was 12.5 wt % based on the total amount of the emulsifier. The molar ratio of the sulfonic acid group to the polyene polyamine skeleton was 0.31:1.

The emulsification rate and demulsification voltage of the emulsifier A2 in standard oil-based emulsions were shown in Table 1.

### Example 3

(1) 20 mol of triethylene tetramine and 45 mol of methyl 9,10-epoxyoctadecanoate (cas: 6084-76-0) were added into a double-neck round-bottom flask with a reflux condenser pipe, the materials were mixed and stirred uniformly, 0.3 mol of phosphoric acid was added and blended, and then stirred and heated to 80° C. for performing the reaction for 6 hours, a main emulsifier was obtained. The molar ratio of the hydrophobic group to the triethylene tetramine skeleton was 2.25:1.
(2) The main emulsifier, 8 mol of sodium 3-chloro-2-hydroxy propane sulfonate and 150 mL of isopropanol were uniformly mixed, 4 mol of potassium carbonate was added and mixed, the mixture was heated to 80° C., stirred and subjected to reaction for 3 hours, a second intermediate was obtained;
(3) The second intermediate was cooled to room temperature, then reduced pressure distilled under the pressure of 1.2 kPa and the temperature of 50° C. to remove solvent isopropanol, an emulsifier A3 was prepared.

The emulsifier A3 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 4.3 wt %, the content of C element was 64.4 wt %, the content of H element was 10.7 wt %, the content of S element was 1.1 wt %, and the content of O element was 15.9 wt %. The molar ratio of S element to N element in the emulsifier was 0.14:1. The content of the triethylene tetramine skeleton was 11.2 wt % based on the total amount of the emulsifier. The molar ratio of the sulfonic acid group to the polyene polyamine skeleton was 0.45:1.

The emulsification rate and demulsification voltage of the emulsifier A3 in standard oil-based emulsions were shown in Table 1.

### Example 4

(1) 20 mol of triethylene tetramine and 52 mol of methyl 9,10-epoxyoctadecanoate (cas: 6084-76-0) were added into a double-neck round-bottom flask with a reflux condenser pipe, the materials were mixed and stirred uniformly, 0.2 mol of para-toluenesulfonic acid was added and blended, and then stirred and heated to 110° C. for performing the reaction for 12 hours, a first intermediate was obtained. The molar ratio of the hydrophobic group to the triethylene tetramine skeleton was 2.6:1.
(2) The first intermediate, 8 mol of sodium 3-chloro-2-hydroxy propane sulfonate and 300 mL of n-octanol were uniformly mixed, 8 mol of potassium carbonate was added and mixed, the mixture was heated to 120° C., stirred and subjected to reaction for 5 hours, a second intermediate was obtained;
(3) The second intermediate was cooled to room temperature, then reduced pressure distilled under the pressure of 1.2 kPa and the temperature of 50° C. to remove solvent n-octanol, an emulsifier A4 was prepared.

The emulsifier A4 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 40 wt %, the content of C element was 65.4 wt %, the content of H element was 11.4 wt %, the content of S element was 0.9 wt %, and the content of O element was 16.8 wt %. The molar ratio of S element to N element in the emulsifier was 0.13:1. The content of the triethylene tetramine skeleton was 9.9 wt % based on the total amount of the emulsifier. The molar ratio of the sulfonic acid group to the polyene polyamine skeleton was 0.39:1.

The emulsification rate and demulsification voltage of the emulsifier A4 in standard oil-based emulsions were shown in Table 1.

### Example 5

The emulsifier was prepared according to the method in Example 1, except that the methyl 9,10-epoxyoctadecanoate (cas: 6084-76-0) was replaced with an equimolar amount of 1,2-epoxy dodecane, an emulsifier A5 was obtained.

The emulsifier A5 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.9 wt %, the content of C element was 67.3 wt %, the content of H element was 11.9 wt %, the content of S element was 1.3 wt %, and the content of O element was 8.9 wt %. The molar ratio of S element to N element in the emulsifier was 0.1:1. The content of the diethylene triamine skeleton was 14.5 wt % based on the total amount of the emulsifier. The molar ratio of the sulfonic acid group to the polyene polyamine skeleton was 0.29:1.

The emulsification rate and demulsification voltage of the emulsifier A5 in standard oil-based emulsions were shown in Table 1.

### Example 6

The emulsifier was prepared according to the method in Example 1, except that the sodium 3-chloro-2-hydroxypropane sulfonate was replaced with an equimolar amount of 1,3-propane sultone, an emulsifier A6 was obtained.

The emulsifier A6 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.4 wt %, the content of C element was 66.1 wt %, the content of H element was 10.8 wt %, the content of S element was 1.3 wt %, and the content of O element was 15.1 wt %. The molar ratio of S element to N element in the emulsifier was 0.11:1. The content of the diethylene triamine skeleton was 13.2 wt % based on the total amount of the emulsifier. The molar ratio of the sulfonic acid group to the polyene polyamine skeleton was 0.32:1.

The emulsification rate and demulsification voltage of the emulsifier A6 in standard oil-based emulsions were shown in Table 1.

### Example 7

The emulsifier was prepared according to the method in Example 1, except that the sodium 3-chloro-2-hydroxypropane sulfonate was used in an amount of 14 mol, an emulsifier A7 was obtained.

The emulsifier A7 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 50 wt %, the content of S element was 2.73 wt %. The molar ratio of S element to N element in the emulsifier was 0.24:1. The content of the diethylene triamine skeleton was 11.9 wt % based on the total amount of the emulsifier. The molar ratio of the sulfonic acid group to the polyene polyamine skeleton was 0.71:1.

The emulsification rate and demulsification voltage of the emulsifier A7 in standard oil-based emulsions were shown in Table 1.

### Example 8

The emulsifier was prepared according to the method in Example 1, except that the sodium 3-chloro-2-hydroxypropane sulfonate was used in an amount of 4.2 mol, an emulsifier A8 was obtained.

The emulsifier A8 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.6 wt %, the content of S element was 0.8 wt %. The molar ratio of S element to N element in the emulsifier was 0.07:1. The content of the diethylene triamine skeleton was 13.3 wt % based on the total amount of the emulsifier. The molar ratio of the sulfonic acid group to the polyene polyamine skeleton was 0.19:1.

The emulsification rate and demulsification voltage of the emulsifier A8 in standard oil-based emulsions were shown in Table 1.

### Comparative Example 1

The emulsifier was prepared according to the method in Example 1, except that steps (2) and (3) were not performed, the main emulsifier obtained in step (1) was used as an emulsifier DAL.

The emulsification rate and demulsification voltage of the emulsifier DA1 in standard oil-based emulsions were shown in Table 1.

### Comparative Example 2

(1) 20 mol of diethylene triamine and 38 mol of methyl 9,10-epoxyoctadecanoate (cas: 6084-76-0) were added into a double-neck round-bottom flask with a reflux condenser pipe, the materials were mixed and stirred uniformly, 0.2 mol of para-toluenesulfonic acid was added and blended, and then stirred and heated to 90° C. for performing the reaction for 7.5 hours, a first intermediate was obtained;
(2) The first intermediate, 22 mol of sodium 3-chloro-2-hydroxy propane sulfonate and 1,000 mL of n-octanol were uniformly mixed, 22 mol of potassium carbonate was added and mixed, the mixture was heated to 100° C., stirred and subjected to reaction for 4 hours, a second intermediate was obtained;
(3) The second intermediate was cooled to room temperature, then reduced pressure distilled under the pressure of 1.2 kPa and the temperature of 50° C. to remove solvent ethanol, an emulsifier DA2 was prepared.

The emulsifier DA2 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 4.1 wt %, the content of S element was 3.4 wt %. The molar ratio of S element to N element in the emulsifier was 0.36:1. The content of the diethylene triamine skeleton was 10 wt % based on the total amount of the emulsifier. The molar ratio of the sulfonic acid group to the polyene polyamine skeleton was 1.09:1.

The emulsification rate and demulsification voltage of the emulsifier DA2 in standard oil-based emulsions were shown in Table 1.

### Comparative Example 3

(1) 20 mol of triethylene tetramine and 40 mol of methyl 9,10-epoxyoctadecanoate (cas: 6084-76-0) were added into a double-neck round-bottom flask with a reflux condenser pipe, the materials were mixed and stirred uniformly, 0.2 mol of para-toluenesulfonic acid was added and blended, and then stirred and heated to 80° C. for performing the reaction for 6 hours, a first intermediate was obtained.
(2) The first intermediate, 40 mol of sodium 3-chloro-2-hydroxy propane sulfonate and 2,000 mL of n-octanol were uniformly mixed, 40 mol of potassium carbonate was added and mixed, the mixture was heated to 120° C., stirred and subjected to reaction for 8 hours, a second intermediate was obtained;
(3) The second intermediate was cooled to room temperature, then reduced pressure distilled under the pressure of 1.2 kPa and the temperature of 50° C. to remove solvent n-octanol, an emulsifier DA3 was prepared.

The emulsifier DA3 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.1 wt %, the content of S element was 5.9 wt %. The molar ratio of S element to N element in the emulsifier was 1.5:1. The content of the triethylene tetramine skeleton was 13.3 wt % based on the total amount of the emulsifier. The molar ratio of the sulfonic acid group to the polyene polyamine skeleton was 2.02:1.

The emulsification rate and demulsification voltage of the emulsifier DA3 in standard oil-based emulsions were shown in Table 1.

### Comparative Example 4

The main emulsifier Span 80 and the auxiliary emulsifier Tween 80 were mixed according to the molar ratio 3:1, the mixture was used as an emulsifier DA4.

The emulsification rate and demulsification voltage of the emulsifier DA4 in standard oil-based emulsions were shown in Table 1.

TABLE 1

| | | Volume | Addition | Emulsi- | Demulsi- |
|---|---|---|---|---|---|
| Emulsifier | Base | ratio of | amount of the | fication | fication |
| Number | oil | oil-water | emulsifier wt % | ratio % | voltage V |
| A1 | 5# | 80:20 | 5 | 100 | 264 |
| | white | | | | |
| | oil | | | | |
| A1 | 5# | 80:20 | 5 | 100 | 274 |
| purified | white | | | | |
| product | oil | | | | |
| A2 | 5# | 80:20 | 5 | 100 | 272 |
| | white | | | | |
| | oil | | | | |
| A3 | 5# | 80:20 | 5 | 100 | 245 |
| | white | | | | |
| | oil | | | | |
| A4 | 5# | 80:20 | 5 | 100 | 238 |
| | white | | | | |
| | oil | | | | |
| A5 | 5# | 80:20 | 5 | 100 | 212 |
| | white | | | | |
| | oil | | | | |
| A6 | 5# | 80:20 | 5 | 100 | 227 |
| | white | | | | |
| | oil | | | | |
| A7 | 5# | 80:20 | 5 | 92 | 125 |
| | white | | | | |
| | oil | | | | |
| A8 | 5# | 80:20 | 5 | 100 | 389 |
| | white | | | | |
| | oil | | | | |
| DA1 | 5# | 80:20 | 5 | 100 | 432 |
| | white | | | | |
| | oil | | | | |
| DA2 | 5# | 80:20 | 5 | 53 | 54 |
| | white | | | | |
| | oil | | | | |
| DA3 | 5# | 80:20 | 5 | 48 | 47 |
| | white | | | | |
| | oil | | | | |
| DA4 | 5# | 80:20 | 5 | 24 | 384 |
| | white | | | | |
| | oil | | | | |

### Test Example

### Preparation of Drilling Fluids:

Based on 100 parts by weight of 5 # white oil, 4 parts by weight of the emulsifiers prepared in the Examples and Comparative Example were added separately, the materials were stirred at high speed of 10,000 rpm for 30 min, the CaCl₂ aqueous solution with a concentration of 25 wt % was then added according to the volume ratio of 80:20. Subsequently, based on 100 parts by weight of 5 # white oil, 4 parts by weight of natural asphalt, 2 parts by weight of calcium oxide, 2 parts by weight of organic bentonite were added, barite was then added to improve the weight and increase the density to the range of 1.8-2.2 g/cm³, the stirring with a high speed was continued for 2 h, the oil-based drilling fluids with a density of 2 g/cm³ were prepared.
(1) The electrical stability test: a probe of an electrical stability tester was placed in the prepared oil-based drilling fluid, the measurement results were recorded, the average demulsification voltage value of the two measurement results were calculated, wherein the difference of the two readings was not more than 5%.

The plastic viscosity (PV), yield point (YP) and high-temperature high-pressure filtrate loss (FLHTHP) of the drilling fluids were tested according to the China National Standard GB/T16783.2-2012 “Petroleum and natural gas industries—Field testing of drilling fluids—Part 2: Oil-based fluids”. The results were shown in Table 2.

TABLE 2

| | Density | | | | Demulsification |
|---|---|---|---|---|---|
| No. | g/cm³ | PV/mPs | YP/Pa | FLHTHP/mL | voltage/V |
| A1 | 2 | 33 | 5.5 | 2.2 | 970 |
| A1 | 2 | 34 | 5.5 | 1.8 | 1089 |
| purified | | | | | |
| product | | | | | |
| A2 | 1.8 | 26 | 4 | 2.2 | 1014 |
| A3 | 2.2 | 39 | 5.5 | 2.6 | 846 |
| A4 | 2 | 32 | 5.5 | 2.4 | 912 |
| A5 | 2 | 35 | 5 | 3 | 712 |
| A6 | 2 | 36 | 4.5 | 3.2 | 689 |
| A7 | 2 | 31 | 5 | 2.6 | 674 |
| A8 | 2 | 38 | 6.5 | 2.8 | 742 |
| DA1 | 2 | 40 | 7 | 4.2 | 501 |
| DA2 | 2 | 30 | 4 | 5.8 | 524 |
| DA3 | 2 | 28 | 4.5 | 6.4 | 572 |
| DA4 | 2 | 38 | 5.5 | 4.4 | 567 |

### (2) Thermal Stability Test

The prepared drilling fluids were subjected to an aging process at 180° C. for 24 h, respectively, the drilling fluid properties were tested according to the same method, the results were shown in Table 3.

The prepared drilling fluids were subjected to an aging process at 200° C. for 96 h, respectively, the drilling fluid properties were tested according to the same method, the results were shown in Table 4.

TABLE 3
After the aging process (180° C./24 h)

| | Density | | | | Demulsification |
|---|---|---|---|---|---|
| No. | g/cm³ | PV/mPs | YP/Pa | FLHTHP/mL | voltage/V |
| A1 | 2 | 26 | 4.5 | 2.8 | 828 |
| A1 | 2 | 25 | 5 | 2.2 | 974 |
| purified | | | | | |
| product | | | | | |
| A2 | 1.8 | 23 | 3.5 | 2.8 | 932 |
| A3 | 2.2 | 35 | 4 | 3 | 725 |
| A4 | 2 | 31 | 5 | 3.4 | 742 |
| A5 | 2 | 24 | 3.5 | 3.6 | 611 |
| A6 | 2 | 27 | 3 | 3.8 | 572 |
| A7 | 2 | 24 | 4 | 2.4 | 691 |
| A8 | 2 | 42 | 7 | 3.2 | 518 |
| DA1 | 2 | 52 | 8.5 | 6.2 | 368 |
| DA2 | 2 | 20 | 2 | 6.8 | 478 |
| DA3 | 2 | 22 | 2 | 7.2 | 420 |

TABLE 4
After the aging process (200° C./96 h)

| | Density | | | | Demulsification |
|---|---|---|---|---|---|
| No. | g/cm³ | PV/mPs | YP/Pa | FLHTHP/mL | voltage/V |
| A1 | 2 | 26 | 2 | 4.2 | 546 |
| A1 | 2 | 24 | 3.5 | 3.2 | 648 |
| purified | | | | | |
| product | | | | | |
| A2 | 1.8 | 23 | 1.5 | 4.8 | 512 |
| A3 | 2.2 | 24 | 2 | 4.4 | 504 |
| A4 | 2 | 24 | 1.5 | 4.4 | 508 |
| A5 | 2 | 24 | 1.5 | 4.8 | 432 |
| A6 | 2 | 26 | 2 | 4.6 | 464 |
| A7 | 2 | 24 | 1.5 | 4.6 | 432 |
| A8 | 2 | 52 | 3.5 | 8.4 | 437 |
| DA1 | 2 | 62 | 10.5 | 12.4 | 312 |
| DA2 | 2 | 16 | 0 | 14.8 | 348 |
| DA3 | 2 | 18 | 0 | 16.8 | 291 |

As can be seen with reference to the results in Tables 2-4, the emulsification ratio and electrical stability of the emulsifier products obtained in Examples of the present invention have significant advantages over the emulsifier products prepared in Comparative Examples.

The above content describes in detail the preferred embodiments of the present disclosure, but the present disclosure is not limited thereto. A variety of simple modifications can be made in regard to the technical solutions of the present disclosure within the scope of the technical concept of the present disclosure, including a combination of individual technical features in any other suitable manner, such simple modifications and combinations thereof shall also be regarded as the content disclosed by the present disclosure, each of them falls into the protection scope of the present disclosure.

1.-16. (canceled)

17. An emulsifier, wherein the emulsifier has a polyene polyamine skeleton and a hydrophobic group connected to the skeleton, and part of the polyene polyamine skeleton is connected to a sulfonic acid group, wherein a molar ratio of a sulfur element to a nitrogen element in the emulsifier is 0.02-0.4:1.

18. The emulsifier according to claim 17, wherein the molar ratio of a sulfur element to a nitrogen element in the emulsifier is 0.08-0.3:1.

19. The emulsifier according to claim 17, wherein a molar ratio of the hydrophobic group to the polyene polyamine skeleton is (1-4):1.

20. The emulsifier according to claim 17, wherein a content of the polyene polyamine skeleton is within a range of 5-20 wt %, based on a total amount of the emulsifier;
and/or, a molar ratio of the sulfonic acid group to the polyene polyamine skeleton is (0.1-1.2):1.

21. The emulsifier according to claim 17, wherein the emulsifier contains C, H, N, S and O elements.

22. The emulsifier according to claim 21, wherein a content of C element is 50-75 wt %, a content of H element is 5-15 wt %, a content of N element is 2-8 wt %, a content of S element is 0.5-2 wt %, and a content of O element is 10-25 wt %, based on a total amount of the emulsifier.

23. The emulsifier according to claim 17, wherein the polyene polyamine skeleton is at least one of diethylene triamine skeleton, triethylene tetramine skeleton and tetraethylene pentamine skeleton;
and/or, the hydrophobic group contains at least one hydrophobic functional group;
and/or, the hydrophobic group is 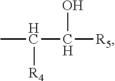
wherein R₄ is selected from H or C5-C16 alkyl;
and R₅ is selected from substituted or unsubstituted C5-C16 alkyl, alkoxy, aryl or phenoxy.

24. The emulsifier according to claim 17, wherein a structure of the sulfonic acid group is represented by 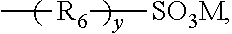
wherein R₆ is selected from substituted or unsubstituted C1-C8 alkylidene; y is 0 or 1; and M is selected from Na, K or ammonium group.

25. The emulsifier according to claim 17, wherein the emulsifier comprises a compound represented by formula (I): 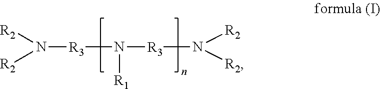
wherein each R₁ is independently selected from H or the sulfonic acid group, each R₂ is independently selected from H or the hydrophobic group, and at least one R₂ is not H; each R₃ is independently selected from substituted or unsubstituted C1-C3 alkylidene; n is a positive integer between 1 and 3.

26. The emulsifier according to claim 17, wherein a demulsification voltage of the emulsifier in a standard oil-based emulsion under condition of a dosage of 5 wt % is within a range of 50-400V, wherein the standard oil-based emulsion is composed of 5 # white oil and CaCl₂) aqueous solution having a concentration of 25 wt %, a volume ratio of oil-water is 80:20;
and/or, an emulsification ratio of the emulsifier in the standard oil-based emulsion is larger than or equal to 90%.

27. A preparation method for an emulsifier, the preparation method comprises the following steps: contacting a main emulsifier with a sulfonating agent to carry out a sulfonation reaction to introduce a sulfonic acid group into a portion of the main emulsifier; wherein the main emulsifier has a polyene polyamine skeleton and a hydrophobic group connected to the skeleton;
a molar ratio of the sulfonating agent to the main emulsifier calculated in terms of polyene polyamine skeleton is (0.1-0.5):1.

28. The preparation method according to claim 27, wherein the molar ratio of the sulfonating agent to the main emulsifier calculated in terms of polyene polyamine skeleton is (0.2-0.4):1;
and/or, conditions for the sulfonation reaction comprise a reaction temperature within a range of 80-120° C. and a time of 3-5 hours;
and/or, the sulfonation reaction is carried out in presence of an acid-binding agent, a molar ratio of the acid-binding agent to the sulfonating agent is (0.5-2.5):1.

29. The preparation method according to claim 27, wherein a molar ratio of the hydrophobic group to the polyene polyamine skeleton is (1-4):1.

30. The preparation method according to claim 27, wherein the polyene polyamine skeleton is at least one of diethylene triamine skeleton, triethylene tetramine skeleton, and tetraethylene pentamine skeleton; and/or,
the hydrophobic group contains at least one hydrophobic functional group;
and/or, the hydrophobic group is 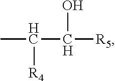
wherein R₄ is selected from H or C5-C16 alkyl;
and R₅ is selected from substituted or unsubstituted C5-C16 alkyl, alkoxy, aryl or phenoxy.

31. The preparation method according to claim 27, wherein the main emulsifier comprises a compound with a structure represented by formula (i): 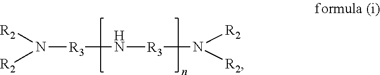
wherein each R₂ is independently selected from H or the hydrophobic group, and at least one R₂ is not H; each R₃ is independently selected from substituted or unsubstituted C1-C3 alkylidene; n is a positive integer between 1 and 3.

32. The preparation method according to claim 27, wherein the sulfonating agent is selected from sulfonate and/or alkyl sulfonate lactone.

33. The preparation method according to claim 32, wherein the sulfonate has a structure represented by formula (ii): 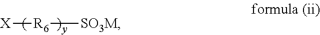
wherein X is a halogen atom; R₆ is selected from substituted or unsubstituted C1-C8 alkylidene; y is 0 or 1; M is selected from Na, K or ammonium group;
and/or, the alkyl sulfonate lactone is selected from C3-C5 alkyl sulfonates.

34. An emulsifier produced with the preparation method according to claim 27.

35. An oil-based drilling fluid, wherein the oil-based drilling fluid comprises a base oil, and a treatment agent comprising the emulsifier according to claim 17.

36. The oil-based drilling fluid according to claim 35, wherein a content of the emulsifier is 2-10 parts by weight, based on 100 parts by weight of the base oil.
